(19) United States
(12) Patent Application Publication
(10) Pub. No.: US 2016/0082246 A1
(43) Pub. Date: Mar. 24, 2016

(54) DEFIBRILLATOR ELECTRODE HAVING ADULT AND PEDIATRIC GRAPHICS

(71) Applicant: KONINKLIJKE PHILIPS N.V., Eindhoven (NL)

(72) Inventor: AARON JAMES PIAZZA, SEATTLE, WA (US)

(73) Assignee: KONINKLIJKE PHILIPS N.V., Eindhoven (NL)

(21) Appl. No.: 14/888,579

(22) PCT Filed: May 8, 2014
(86) PCT No.: PCT/IB2014/061287

§ 371 Date: Nov. 2, 2015

Related U.S. Application Data
(60) Provisional application No. 61/821,269, filed on May 9, 2013.

(51) Int. Cl. A61N 1/04; A61N 1/39

(52) U.S. Cl. CPC A61N 1/046; A61N 1/0492; A61N 1/3912

(57) ABSTRACT

An electrode pad (110, 210) for a defibrillator has an improved graphic indication for placement of the pad on a patient during a rescue. The graphic indication may include both of an adult figure and a pediatric figure showing the placement of the electrode pad. Thus, the graphic figures assist the rescuer in placing the electrode pad for both adult and pediatric patients.

The present invention relates generally to an electrotherapy apparatus and method for delivering a series of shocks to a patient's heart. More particularly, this invention pertains to an improved Automated External Defibrillator (AED) and electrode set that is suitable for defibrillating patients of all ages.

Sudden Cardiac Arrest (SCA) is one of the leading causes of death in the industrialized world, and typically results from an arrhythmia condition known as Ventricular Fibrillation (VF), during which a patient's heart muscle exhibits extremely rapid, uncoordinated contractions that render the heart incapable of circulating blood. Statistically, after the first four minutes, the patient's chance of survival decreases by approximately 10% during each subsequent minute they fail to receive treatment.

An generally effective treatment for VF is electrical defibrillation, in which a defibrillator delivers an electrical pulse or shock to the patient's heart. Because the onset of VF is generally an unpredictable event, the likelihood that a victim will survive rises dramatically if defibrillation equipment is nearby. As a result, medical equipment manufacturers have developed Automated External Defibrillators (AEDs) that minimally trained personnel may employ to perform electrical defibrillation when emergency situations arise. AEDs may be found in non-medical settings such as residences, public buildings, businesses, private vehicles, public transportation vehicles, and airplanes, for example.

To increase a patient's chances of survival, AED operators generally must perform quickly and accurately in life-threatening situations. Hence, AEDs are typically designed to be simple and intuitive to use. AEDs often automate many of the steps associated with operating external defibrillation equipment, and minimize the number of decisions the operator must make. An AED may provide voice instructions or commands to guide the operator through application of the device. An AED may also analyze a patient's heart rhythm to determine when administration of an electrical shock to the patient may be appropriate. If a shock is warranted, the AED facilitates delivery of a defibrillation waveform at a particular energy level.

Some defibrillators have included graphics on the electrode pads that are applied to the patient for delivering electrotherapy. One co-assigned patent, U.S. Pat. No. 5,951,598 entitled “Electrode system”, the entire disclosure of which is incorporated herein by reference, describes, e.g., a printed graphic on each electrode which shows an image of at least a portion of an adult human body, further having an image of the proper position of the electrode pad on the body image.

The large majority of VF situations involve adult patients, as VF tends to be a relatively rare condition in children and/or infants. Nonetheless, recent evidence suggests that pediatric VF occurs with sufficient frequency to be of concern. AEDs, however, are generally designed for use on/with adults. Further, in the past, pediatric application of AEDs had generally been limited by a lack of data characterizing pediatric Electrocardiogram (ECG) rhythms, which generally cast doubt upon the effectiveness of EGG detection algorithms that an AED may employ in pediatric situations.

Additionally, energy delivery recommendations for children are dependent upon body mass, whereas such recommendations for adults generally are not. Presently, the recognized treatment for pediatric VF in children less than 8 years of age is manual defibrillation in which delivered energies are proportional to the patient's body weight (e.g., 1 Joule per kilogram of body weight, increasing to 2 Joules per kilogram if necessary). Incorporating controls to facilitate detailed energy adjustments in accordance with body mass or weight would generally add extra complexity and cost to an AED design. Moreover, providing such controls could/would undesirably complicate the decisions operators must make during time-critical situations, even when treating adults, thereby providing more opportunity for treatment to fail and generally decreasing the patient's chance of the survival.

Some defibrillators may include an adult/pediatric mode control or switch. Based upon a setting of the adult/pediatric switch, the AED may perform an adult defibrillation sequence or a pediatric defibrillation sequence upon a patient. An adult defibrillation sequence may involve the delivery of one or more defibrillator shocks characterized by an energy appropriate for adults. For example, an adult defibrillation sequence may comprise the delivery of one or more 150 Joule biphasic waveforms to the patient, whereas a pediatric defibrillation sequence may involve the delivery of one or more defibrillator shocks characterized by an energy appropriate for children who are or seem to be less than eight years of age. For example, a pediatric defibrillation sequence may comprise the delivery of one or more 50 Joule biphasic defibrillator shocks to the patient.

Furthermore, a defibrillator such as described above may employ a universal electrode suitable for use on all patients that is smaller than a conventional adult electrode, yet larger than a conventional pediatric electrode. For example, such a defibrillator and electrode are described in co-assigned U.S. Pat. No. 7,062,321 entitled “Method and apparatus for defibrillating patients of all ages”, the entire disclosure of which is incorporated herein by reference.

Graphics that appear on existing electrodes, however, generally restrict their placement guidance to either adult or pediatric patients, e.g., in order to minimize confusion during the much-more-common adult cardiac rescues. But such graphics simplify the electrode placement only if used in situations matching the patient, and can cause/induce confusion or delay in situations where the graphic type and the patient differ from one another. For example, if a universal electrode is used on a pediatric patient, but has only adult placement markings, the rescuer must recollect prior training or take the time to look up the proper placement, e.g., in an instruction manual to know that the preferred placement generally is anterior-posterior instead of the illustrated apex-sternum adult placement. This problem can be especially exacerbated in defibrillators having a mode switch.

What is needed therefore is an improved user guidance illustration for a universal electrode, which provides electrode placement guidance for patients of all ages.

In accordance with the objectives of the present invention, the inventors have invented and developed, e.g., a graphic illustration which intuitively guides the user to correct placement in both the adult and pediatric cardiac rescues. By inventively arranging both adult and pediatric graphics on each of the electrodes, guidance is provided for both types of rescue. Furthermore, by inventive sizing, orientation, and coloring of the graphics, exemplary embodiments of the present invention can provide the appropriate amount of information and guidance while avoiding confusion, distraction and time delays that could otherwise be caused.

In accordance with the objectives of the invention, an exemplary embodiment of an electrode for a defibrillator is described comprising a lead wire, a substrate, and a conductive surface disposed on one side of the substrate and electrically connected to the lead wire. The other graphics side of each electrode includes a printed graphic showing the proper positioning of the electrode on a patient torso, wherein the graphic includes both a figure of an adult body and of a pediatric body.

In accordance with another aspect of the invention, an exemplary embodiment of an external defibrillator is described comprising a high voltage energy source structured, configured and operable to deliver electrotherapy, an electrode connector electrically connected to the high voltage energy source, a controller, and a mode switch operable to switch the controller from an adult defibrillation mode to a pediatric defibrillation mode. In accordance with exemplary embodiments of the present invention, the defibrillator can also include an electrode storage device having a release liner surface, and first and second universal electrode pads disposed on the release liner surface, each electrode pad having a lead wire connected to the electrode connector, a printable surface, and a graphic indication disposed on the printable surface of the proper placement of the respective electrode pad on both of an adult human body and a pediatric human body.

In accordance with yet another aspect of the invention, an exemplary embodiment of a medical electrode system is described comprising first and second electrode pads having a lead wire electrically connected to each electrode pad. Each of the first and second electrode pads comprises a patient-facing side adapted to be placed on a patient and a graphics side adapted to be visible when the patient-facing side is placed on a patient. The graphics side of the first electrode pad comprises, e.g., a first image of at least a portion of an adult human body showing a proper adult placement of the first electrode pad, and a second image of at least a portion of a pediatric human body showing a proper pediatric placement of the first electrode pad. The graphics side of the second electrode pad comprises, e.g., a third image of at least a portion of an adult human body showing a second proper adult placement of the second electrode pad, and a fourth image of at least a portion of a pediatric human body showing a second proper pediatric placement of the second electrode pad.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

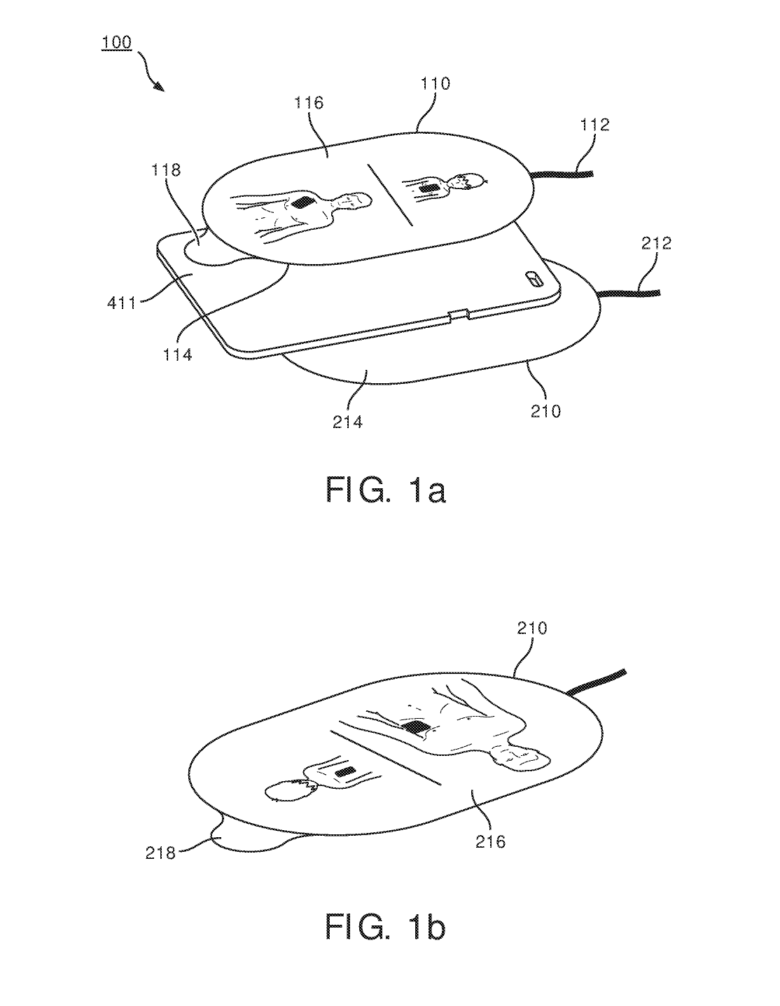

FIGS. 1a and 1b illustrate a medical electrode system according to an exemplary embodiment of the present invention.

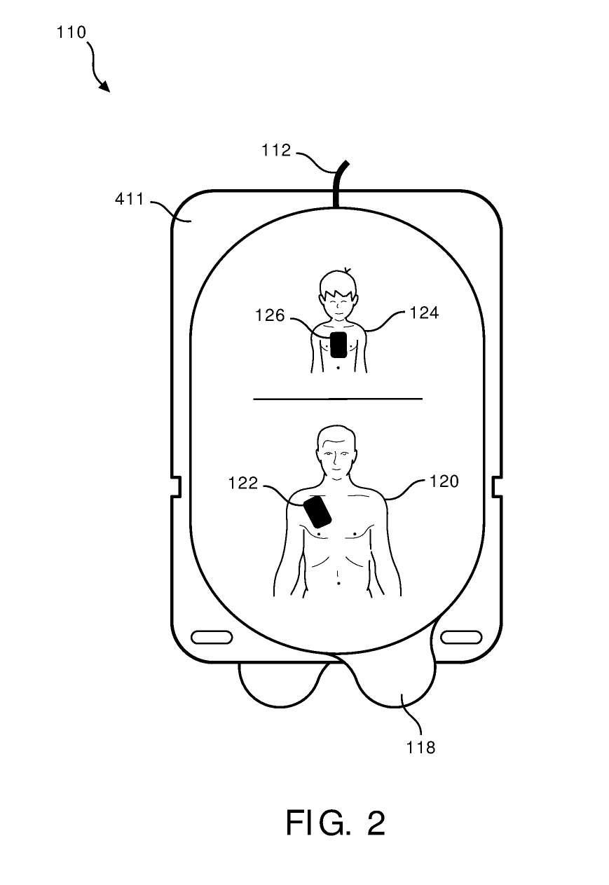

FIG. 2 illustrates a graphic indicator on one medical electrode pad according to an exemplary embodiment of the present invention.

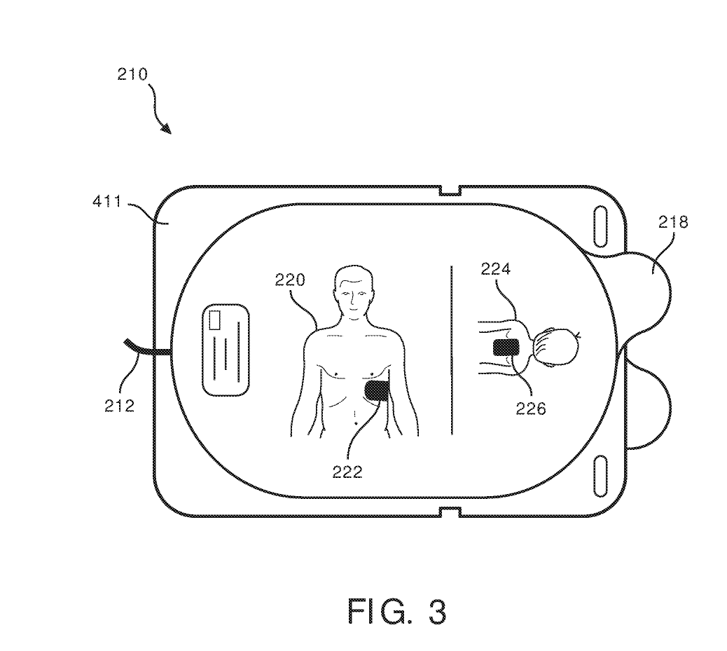

FIG. 3 illustrates a graphic indicator on another medical electrode pad according to an exemplary embodiment of the present invention.

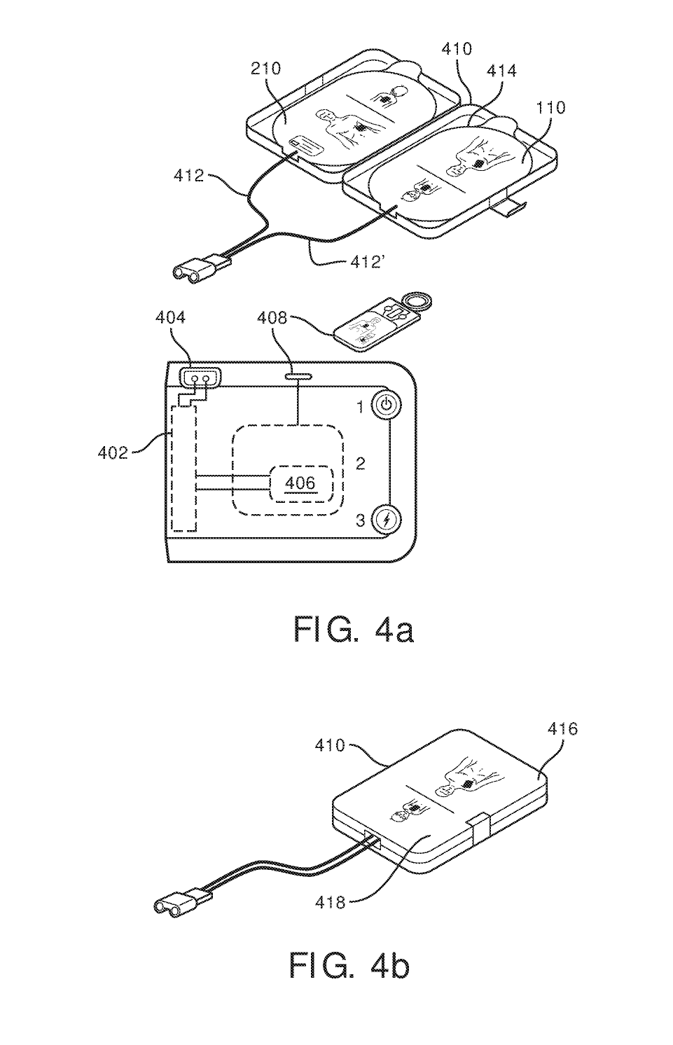

FIGS. 4a and 4b illustrate a defibrillator including electrodes according to an exemplary embodiment of the present invention.

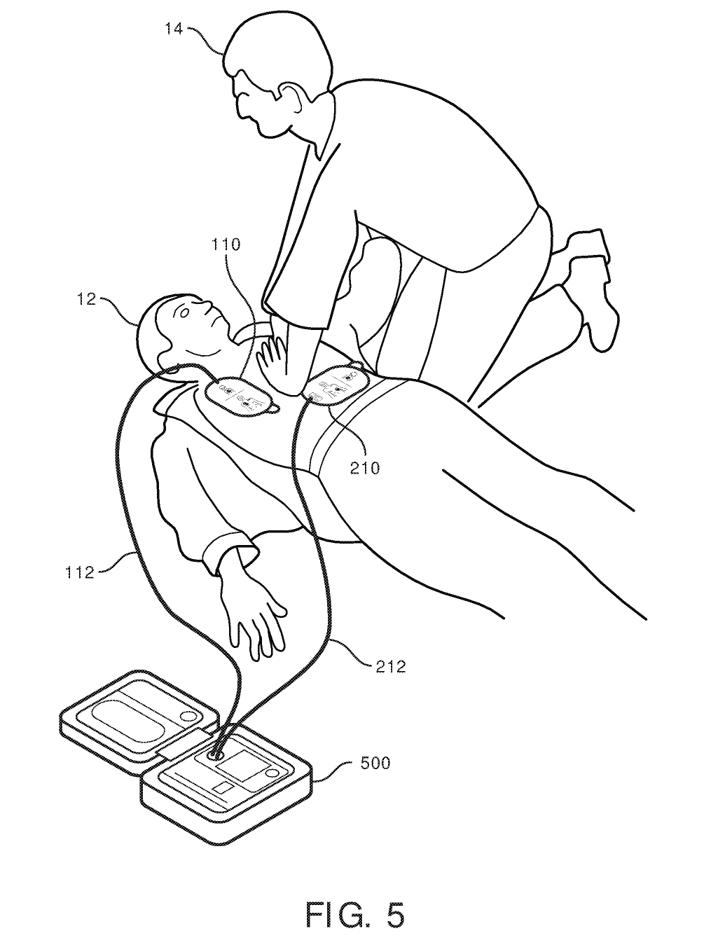

FIG. 5 illustrates an exemplary embodiment of the present invention in use during a cardiac rescue, in accordance with the present disclosure.

Referring first to FIG. 1a, a medical electrode system 100 is illustrated according to an exemplary embodiment of the present invention. The arrangement of the electrode system 100 can be similar in some respects to the electrode illustrates in co-assigned U.S. Patent Publication 2007/0203558 entitled “Self-storing Medical Electrodes”, the contents of which is incorporated by reference. Shown in FIG. 1a are two electrode pads 110, 210 disposed on a central release liner sheet 411, which is hereafter referred to as a hardliner.

Hardliner 411 is a substantially rigid sheet having a release liner surface on both sides. The two electrode pads 110, 210 are disposed on either side of the hardliner 411 with the patient-facing side of each pad in a face-to-face relationship.

Each electrode pad 110, 210 includes a respective lead wire 112, 212 which is electrically attached to a conductive surface disposed on a substrate, the conductive surface being on a patient-facing side 114 or 214. The conductive surface may include a conductive gel for adhering to the patient's skin, and for conveying defibrillation energy from the electrode to the patient. Each electrode patient-facing side is disposed on a release surface on each side of hardliner 411 for storage. In order to facilitate the deployment of the electrode pads 110,210 from hardliner 411, each electrode may include a respective peel tab 118, 218.

As shown in FIG. 1a, the other side of the first electrode pad 110 substrate, referred to as the graphics side 116, includes visual indications which are intended to guide the user to place the electrode properly on the patient. Graphics side 116 is preferably a printable surface. Similarly, as shown in FIG. 1b, the second electrode pad 210 patient-facing graphics side 216 includes visual indications which are intended to guide the user to place the second electrode properly on the patient.

By placing the graphic on the electrode itself, the rescuer need only refer to the electrode in hand during the placement procedure. Thus, the rescuer does not have to look back and forth to other instructions during the rescue.

FIG. 2 illustrates a more detailed view of the graphics side 116 of first electrode pad 110, as it resides on a hardliner 411. Graphics side 116 includes a first image 120 of at least a portion of an adult human body, on which is shown a proper adult placement 122 for that electrode pad 110. In this embodiment, the first electrode pad 110 is shown in a sternum position on the first image 120.

Also included on graphics side 116 is a second image 124 of at least a portion of a pediatric human body showing a proper pediatric placement 126 of the first electrode pad 110. In this embodiment, the first electrode pad 110 would be shown in an anterior position on the second image 124. Preferably, the relative size of the pediatric human body image 124 to the adult human body image 126 is in proportion to the actual size of an adult and a pediatric human, in order to further clarify the proper placement, and to emphasize the electrode placement in the more-common adult cardiac rescue.

The images of the adult human body and the pediatric human body may be two dimensional or three dimensional renderings. Additional, the images may include anatomical markers that assist the user in proper placement of the electrode pad 110. For example, depictions of the rib line, the clavicle bone (or collar bone), or the definition of the pectoral muscles may be shown. Additionally, the images of the human body preferably appear as the mirror image of the defibrillator operator, which corresponds to the positioning of the patient when the electrode pads are applied by the operator during treatment.

The adult placement 122 and/or pediatric placement 126 may be of a distinguishing color, pattern, or contrast with the underlying figure graphic 120, 124. The relative size of the placement graphics may be varied somewhat, and may be chosen to show a precise desired position, an acceptable zone of placement on the patient body, or an area somewhere in between.

Although not shown here, arrows, circles, or other features intended to direct the rescuer's attention to the placement instruction may be provided. Also not shown is an additional optional graphic which shows the proper placement of the other, second, electrode placement on the first image 120. This optional graphic would preferably be printed in a less conspicuous emphasis, such as in a lighter weight or a dashed line.

The electrode pad 110 of FIG. 2 shows both of the first image 120 and the second image 124 to be oriented such that each image appears upright with a long dimension of the pad 110 to the left, i.e. in a portrait orientation. Thus, with reference to FIG. 5, each graphic image is oriented on the printable surface in the same orientation as to an intended placement of the respective electrode on the patient 12, such that the rescuer 14 is assured that the electrode is correctly positioned without the need for any other reference. Thus, the rescuer 14 can direct his concentration to the time-critical step of applying the defibrillation electrodes. This allows the connected defibrillator 500 to deliver electrotherapy via the electrodes if necessary.

FIG. 2 also illustrates the orientation of the lead wire 112 with respect to the electrode pad 110 and graphics. As shown, lead wire 112 is electrically connected to the first electrode pad 110 on a short dimension of the pad, and in particular above the heads of the adult and pediatric body images 120, 124. In reference again to FIG. 5, it can be seen that this lead wire disposition enables a “wires out” deployment when the electrode pad 110 is placed as intended on the adult patient 12. In the “wires out” orientation, the lead wire extends away from the center of the torso for unobstructed access to the chest for CPR compressions and such.

Electrode pad 110 may further comprise a peel tab 118 disposed at one end of the electrode. Preferably, peel tab 118 has a distinguishing color or marking for guiding the user to grasp the peel tab for deployment and positioning the electrode 110 to the patient.

FIG. 3 illustrates a more detailed view of the graphics side 216 of second electrode pad 210 as it resides on a hardliner 411. Graphics side 216 includes a third image 220 of at least a portion of an adult human body, on which is shown a proper adult placement 222 for that electrode pad 210. In this embodiment, the second electrode pad 210 is shown in an apex position on the third image 220.

Also included on graphics side 216 is a fourth image 224 of at least a portion of a pediatric human body showing a proper pediatric placement 226 of the second electrode pad 210. In this embodiment, the second electrode pad 210 would be shown in a posterior position on the fourth image 224. Preferably, the relative size of the pediatric human body image 224 to the adult human body image 226 is in proportion to the actual size of an adult and a pediatric human, in order to further clarify the proper placement, and to emphasize the electrode placement in the more-common adult cardiac rescue.

The images of the adult human body and the pediatric human body may be two dimensional or three dimensional renderings. Additional, the images may include anatomical markers that assist the user in proper placement of the electrode pad 210. For example, depictions of the rib line, the clavicle bone (or collar bone), or the definition of the pectoral muscles may be shown. Additionally, the images of the human body preferably appear as the mirror image of the defibrillator operator, which corresponds to the positioning of the patient when the electrode pads are applied by the operator during treatment.

The adult placement 222 and/or pediatric placement 226 may be of a distinguishing color, pattern, or contrast with the underlying figure graphic 220, 224. The relative size of the placement graphics may be varied somewhat, and may be chosen to show a precise desired position, an acceptable zone of placement on the patient body, or an area somewhere in between.

Although not shown here, arrows, circles, or other features intended to direct the rescuer's attention to the placement instruction may be provided. Also not shown is an additional optional graphic which shows the proper placement of the other, first, electrode placement on the third image 220. This optional graphic would preferably be printed in a less conspicuous emphasis, such as in a lighter weight or a dashed line.

The electrode pad 210 of FIG. 3 shows the third image 220 to be oriented such that the adult body appears upside down with a short dimension of the pad 210 to the left, i.e. in a landscape orientation. Thus, with reference to FIG. 5, the adult body graphic image is oriented on the printable surface in the same orientation as to an intended placement of the respective electrode on the adult patient 12, such that the rescuer 14 is assured that the electrode is correctly positioned without the need for any other reference. Thus, the rescuer 14 can direct his concentration to the time-critical step of applying the defibrillation electrodes.

The fourth image 224 of the pediatric patient is oriented on the electrode pad 210 rotated about a quarter turn clockwise from the third image 220 orientation. This orientation corresponds to the same orientation as to an intended placement of the pad 220 on the actual pediatric patient, i.e. vertical orientation on the posterior of the pediatric patient. Again, this orientation assists the rescuer 14 in quickly and accurately placing the electrode 210 on the pediatric patient.

FIG. 3 also illustrates a preferred the orientation of the lead wire 212 with respect to the electrode pad 210 and graphics. As shown, lead wire 212 is electrically connected to the second electrode pad 210 on a short dimension of the pad, and in particular to the left side of the adult body image 220. In reference again to FIG. 5, it can be seen that this lead wire disposition leads to a “wires in” deployment when the electrode pad 210 is placed as intended on the adult patient 12. The inventor has discovered that this disposition for the electrode when mounted at the apex position keeps the lead wire clear of the chest center in most situations.

It is contemplated, however, that a “wires out” orientation, wherein the lead wire extends away from the center of the torso for unobstructed access to the chest for CPR compressions and such, could alternatively be adopted for the second electrode pad 210. This arrangement can be accomplished by inverting the adult human body image 220 on the second electrode 210.

Electrode pad 210 may further comprise a peel tab 218 disposed at one end of the electrode. Preferably, peel tab 218 has a distinguishing color or marking for guiding the user to grasp the peel tab for deployment and positioning the electrode 210 to the patient. As can be seen in FIGS. 2 and 3, the peel tabs 118, 218 are preferably oriented at the same end of the hardliner 411, but offset somewhat for ease of use.

FIGS. 4a and 4b illustrate another embodiment of the invention utilizing the electrode system 100 as previously described with a portable external defibrillator 400. In this embodiment, a clamshell case 410 is employed to store electrodes 110, 210 prior to use as an alternative to hardliner 411. The clamshell case 410 has a release liner surface 414 on the interior to which the patient-facing side of each of the electrode pads is attached.

FIG. 4b illustrates the clamshell case 410 in the closed position, enclosing the electrodes inside. In order to further enhance the placement guidance graphics, it is preferred that at least one of the graphics from the pad graphics side 116, 216 is reproduced on a clamshell graphic 418 disposed on the clamshell exterior surface 416. The clamshell graphic 418 is preferably oriented the same as the graphic on the enclosed electrode pad. Thus placement guidance to the rescuer 14 begins even before the clamshell case 410 is opened.

Returning to FIG. 4a, a defibrillator 400 is shown having a high voltage energy source 402 which delivers electrotherapy to the patient 12 via an electrode connector 404, lead wires 412,412′ and electrodes 110, 210 to complete a circuit. First and second universal electrode pads 110, 210 are shown in the stored position. It is preferred that the electrodes 110, 210 are pre-connected to the electrode connector 404 during storage to facilitate even more rapid deployment in an emergency.

A controller 406 controls the electrotherapy circuit, and in particular adjusts the electrotherapy parameters via an adult/pediatric mode switch 408. The mode switch is operable to cause the controller to switch the defibrillator between an adult defibrillator mode and a pediatric mode. The mode switch 408 in this embodiment is a key, known as a pedi-key, which when inserted into a corresponding slot in defibrillator 400 by the user, causes controller 406 to switch the device into a pediatric operating mode. The pediatric operating mode generally entails delivery of a reduced-energy defibrillation shock.

Several variations within the scope of the afore-described invention will readily occur to those skilled in the art. For instance, the hardliner may be substituted for the clamshell storage in the defibrillator system. The graphics may also vary in design elements somewhat, as long as they obtain the objects of the invention.

Table of Elements

| | |
|---|---|
| 12 | patient |
| 14 | rescuer |
| 100 | medical electrode system |
| 110 | first electrode pad |
| 112 | first lead wire |
| 114 | first electrode pad patient-facing side |
| 116 | first electrode pad graphics side |
| 118 | first electrode pad peel tab |
| 120 | first image of adult |
| 122 | adult position image for first electrode pad |
| 124 | second image of pediatric patient |
| 126 | pediatric position image for first electrode pad |
| 210 | second electrode pad |
| 212 | second lead wire |
| 214 | second electrode pad patient-facing side |
| 216 | second electrode pad graphics side |
| 218 | second electrode pad peel tab |
| 220 | third image of adult |
| 222 | adult position image forsecibd electrode pad |
| 224 | fourth image of pediatric patient |
| 226 | pediatric position image for second electrode pad |
| 400 | external defibrillator |
| 402 | high voltage energy source |
| 404 | electrode connector |
| 406 | controller |
| 408 | mode switch |
| 410 | clamshell case |
| 411 | hardliner |
| 412 | lead wire |
| 412′ | lead wire |
| 414 | clamshell release liner surface |
| 416 | clamshell exterior surface |
| 418 | clamshell graphic |
| 500 | defibrillator |

1. A medical electrode system comprising:
first and second electrode pads; and
a lead wire electrically connected to each electrode pad,
wherein each of said first and second electrode pads comprises a patient-facing side adapted to be placed on a patient and a graphics side,
and further wherein the graphics side of the first electrode pad comprises
a first image of at least a portion of an adult human body showing a proper adult placement of the first electrode pad, and
a second image of at least a portion of a pediatric human body showing a proper pediatric placement of the first electrode pad,
and further wherein the graphics side of the second electrode pad comprises
a third image of at least a portion of an adult human body showing a second proper adult placement of the second electrode pad, and
a fourth image of at least a portion of a pediatric human body showing a second proper pediatric placement of the second electrode pad.

2. The medical electrode system of claim 1
wherein each of the proper adult placement of the first electrode pad in the first image, the proper pediatric placement of the first electrode pad in the second image, the second proper adult placement of the second electrode pad in the third image, and the second proper pediatric placement of the second electrode pad in the fourth image have a distinguishing printing.

3. The medical electrode system of claim 2 wherein the first image further comprises an image of the second proper adult placement of the second electrode pad and the third image further comprises an image of the proper adult placement of the first electrode pad.

4. The medical electrode system of claim 1 wherein the proper pediatric placement of the first electrode pad is an anterior position on the second image, and wherein the second proper pediatric placement of the second electrode pad is a posterior position on the fourth image.

5. The medical electrode system of claim 4, wherein each of the images of at least a portion of the pediatric human body is sized in proportion to each of the images of at least a portion of the adult human body relative to the actual size of an adult and a pediatric human.

6. The medical electrode system of claim 1, wherein each of the first image, second image, third image and fourth image is oriented on the graphics side of the respective electrode pad such that the image body is aligned with the patient when the respective electrode pad is adhered in the correct position on the patient.

7. The medical electrode system of claim 6, wherein a long dimension of the first electrode pad is oriented to the left of the first image and the second image, and further wherein a short dimension of the second electrode pad is oriented to the left of the third image, and further wherein a long dimension of the second electrode pad is oriented to the left of the fourth image when each of the first, second, third and fourth images are viewed in an upright position.

8. The medical electrode system of claim 7, wherein the first electrode pad lead wire is connected to the first electrode pad at a short dimension edge above the first and second images as viewed in the upright position.

9. The medical electrode system of claim 1 further comprising a peel tab disposed at one end of each electrode pad, and further wherein each peel tab has a distinguishing color.

10. The medical electrode system of claim 1, further comprising a clamshell case with an exterior surface and a release liner disposed on an interior surface of the case, wherein the patient-facing side of each of the first and second electrode pads is attached to the release liner, and further wherein each of the first image, second image, third image, and fourth image is disposed on the exterior surface in the same orientation as the respective image on the attached electrode pads.

11. An external defibrillator comprising:
a high voltage energy source operable to deliver electrotherapy;
an electrode connector electrically connected to the high voltage energy source;
a controller;
a mode switch operable to switch the controller from an adult defibrillation mode to a pediatric defibrillation mode;
an electrode storage device having a release liner surface; and
first and second universal electrode pads disposed on the release liner surface, each electrode pad having
a lead wire connected to the electrode connector,
a printable surface, and
a graphic indication disposed on the printable surface of the proper placement of the respective electrode pad on both of an adult human body and a pediatric human body.

12. The external defibrillator of claim 11, wherein the electrode storage device is a clamshell having an interior release liner surface, and further wherein at least one of the graphic indications is reproduced on an exterior surface of the clamshell.

13. The external defibrillator of claim 11, wherein the electrode storage device is a sheet having the release liner surface on both sides, and further wherein each of the first and second universal electrode pads are disposed in a face to face relationship on opposite sides of the sheet.

14. The external defibrillator of claim 11, wherein the mode switch is disposed as a key which is inserted into a corresponding slot in the defibrillator.

15. The external defibrillator of claim 11, wherein each graphic indication is oriented on the printable surface in the same orientation as to an intended placement of the respective electrode on the adult and pediatric patient.

16. The external defibrillator of claim 15, wherein the first universal electrode pad is disposed for attaching to the sternum position on an adult patient, and further wherein the first universal electrode pad lead wire is attached to the first universal electrode pad at a point that is away from the center of the adult patient torso when the first universal electrode pad is placed as intended on the adult patient.

17. An electrode for a defibrillator, comprising:
a lead wire;
a substrate;
a conductive surface disposed on one side of the substrate and electrically connected to the lead wire; and
a printed graphic disposed on a graphics side of the substrate showing the proper positioning of the electrode on a patient torso,
wherein the graphic includes both of a figure of an adult body and a pediatric body.

18. The electrode of claim 17, wherein the printed graphic is arranged on the graphics side such that the orientation of the patient torso graphic is the same as the orientation of the patient when the electrode is properly positioned.

19. The electrode of claim 17, wherein the lead wire extends from the substrate in a wires-out configuration.

20. The electrode of claim 17, wherein the printed graphic further comprises an image of the electrode on each of the figure of the adult body and the pediatric body in a distinguishing color.
